(19) United States
(12) Patent Application Publication
(10) Pub. No.: US 2009/0030343 A1
(43) Pub. Date: Jan. 29, 2009

(54) Process for the production of interchangeable vacuum test tube holders for taking blood samples and product obtained therefrom

(76) Inventors: Alfonsa Casarotto, Rovigo (IT); Chiara Dall'Ara, Masera' (IT)

Correspondence Address: Themis Intellectual Property Counsel, 7660 Fay Ave Ste H535, La Jolla, CA, 92037, US

(21) Appl. No.: 10/595,676

(22) PCT Filed: Nov. 4, 2003
(86) PCT No.: PCT/IT03/00713

§ 371 Date: Feb. 21, 2007

(51) Int. Cl. A61B 5/15; B23P 11/00

(52) U.S. Cl. 600/576; 29/428

(57) ABSTRACT

A process for the production of a tubular holder for vacuum test tubes and the holder obtained therefrom. The process includes the steps of inserting a straight needle into the holder and the subsequent bending of the needle, so that the outer end of the needle is first connected to the edge of a slanted support wall extending from the casing and has a terminal portion aligned with a side wall of the casing, thus facilitating the insertion of the needle into the limb of a patient. The new disposable holder is particularly useful for blood sampling because it provides for the alignment of the needle with respect to the patient's vein.

### BACKGROUND OF THE INVENTION

1. Field of the Invention

The present invention concerns a device for taking blood samples, and, more particularly, a holder provided with a needle, into which holder vacuum test tubes for taking blood samples are inserted, and the process for the production thereof.

2. Description of Related Art

Numerous medical diagnoses require the analysis of blood samples taken directly from a patient's vein.

When blood samples are taken, it is very important to prevent the transmission, both between patients and between patient and nurse, of any diseases, germs, viruses and similar.

Further, the nurse must not insert the needle through the vein, penetrating-the vein from side to side, because this would produce a hematoma in the patient and also because that would make it impossible to take the blood sample.

The syringe, as a means of taking blood samples from a patient, may be replaced with a holder provided with a needle, integral with such holder, and with various vacuum test tubes that are inserted into the holder each time.

The holder consists of a plastic cylinder with the rear end open, and with the front end closed by a circular wall penetrated by the needle.

Said cylinder, which is suited to contain a vacuum test tube, has the disadvantage of being very wide.

The portion of the needle extending out of the holder is inserted in the vein from which the blood sample is to be taken, while the opposite tip of the needle, which is housed inside the holder, pierces the seal cap of the vacuum test tube inserted in the holder, thus conveying the blood inside said vacuum test tube.

The holders with a needle with two opposite tips that are currently known have the needle positioned centrally, or coaxially, with respect to the holder.

During the taking of a blood sample, when the needle extending from the holder is inserted into the patient's vein, the needle penetrates the skin at a very wide angle, because the thickness of the holder makes it impossible to position the needle near the skin in a substantially tangential position. The nurse is therefore forced to contrive various solutions, such as raising the patient's skin or pressing the holder hard against the patient's skin. All of this does not prevent possible perforation of the vein, however. Furthermore, said contrivances cause discomfort and in some cases even pain to the patient.

Since the tip of the needle is slanted or lance-shaped, for better insertion into the skin and into a vein, each time the nurse must check the position of the tip of the needle, which must have its oblique side facing upwards for correct insertion into the skin and vein.

Some nurses use butterfly needles, i.e. small needles provided at the sides with two flat flexible grips and provided with a connection tube connected to a blood container or to a syringe. These butterfly needles simplify insertion into the vein and make the blood sampling less painful, but at the same time the nurse must prepare the butterfly needle, connect it to the container or syringe, insert the butterfly needle into the vein and keep it in position with her/his hand or an adhesive bandage throughout the sampling operation. Furthermore, butterfly needles are expensive to purchase and dispose of.

Holders exist that are provided with a non-central needle, i.e. a needle positioned near the side wall of the holder and parallel to it. These holders are described in U.S. Pat. No. 5,938,622.

Although these holders theoretically enable a better insertion of the needle into the vein, they are expensive and difficult to produce. The related vacuum test tubes, in addition to being expensive to purchase, must be inserted correctly in the holder, i.e. with the eccentric pierceable area perfectly aligned with the needle tip inside the holder.

Holders with the needle bent at two points, i.e. approximately S-shaped with the two end parts parallel and a slanted intermediate part, are also known.

The part of the needle inside the holder is generally in a central or coaxial position with respect to the holder, whereas the outer part of the needle is eccentric and out of line. Said holders with needle bent at two points are described in U.S. Pat. No. 3,520,292.

The production of said holders with needle bent at two points requires the pre-bending of the needle, the creation around its slanted part of the circular wall which provides the bottom of the holder and, lastly, the creation of the cylindrical wall of the holder.

All said work phases involve lengthy production times, the need to position and align the various parts, the possibility of inaccuracy and imperfect joining of the various parts. Consequently, the production of said holders with a bent needle is very costly.

Further, said holders with a bent needle are not convenient for use, among others due to the fact that their production process does not provide for the outer part of the needle to be coordinated with the outer edge of the casing.

The distance of the needle from the edge of the casing, albeit shorter than the distance existing in the holders with a central needle, limits the ability to insert the needle into the vein and all the sampling operations.

### BRIEF SUMMARY OF THE INVENTION

In order to eliminate the above-mentioned disadvantages, a new process has been designed for the production of holders with a needle bent at two points and which can be used with interchangeable vacuum test tubes for taking blood samples.

An objective of the new process is to produce a holder with a needle bent at two points with only a few simple steps.

A further objective of the new process is to produce a holder with a needle bent at two points and having the outer part of the needle essentially aligned with the outer edge of the casing, that is, with a much narrower or nil penetration angle.

A further objective of the new process is to produce a holder with a needle bent at two points without inaccuracies, imperfections, non alignments or incorrect orientation.

A further objective of the new holder with a needle bent at two points is to facilitate the insertion of the needle into the vein without causing the patient discomfort or pain.

A further objective of the new holder is to facilitate the insertion of the holder needle into the vein with no need for the nurse to check the position of the tip of the needle.

A further objective of the new holder is to facilitate the insertion of the holder needle into the vein with no need for the nurse to raise the patient's skin.

A further objective of the new holder is to facilitate the insertion of the holder needle into the vein, avoiding the risk of perforating the vein from side to side.

A further objective of the new holder is to permit the use of common vacuum test tubes provided with seal cap suitable for central perforation.

These and other advantages are achieved through the implementation of the new process for the production of holders with a needle bent at two points, which holders can be used with interchangeable vacuum test tubes for taking blood samples, and through the product obtained therefrom.

The new holder, which is preferably of the disposable type, comprises a substantially cylindrical casing closed at one end, designed to receive vacuum test tubes with a seal cap adapted for a central perforation, and a shaped needle, preferably consisting of two parallel straight sections not lying on the same axis, joined by a further slanted or generically S-shaped section. Said needle is joined to the casing so that its portion inside the casing is coaxial with said casing, and so that its portion outside the casing and facing the same is shaped and, in its end part, parallel and eccentric to the axis of the casing, while an outer support wall or front connection wall of the casing acts as a thrust bearer against the needle.

The casing features a wall designed to house and join the angled, bent or shaped section of the needle to the casing.

The holder has the additional advantage of being disposable, thus avoiding all health risks for the users.

The process for the production of the new holder with needle bent at two points includes the complete production of the casing with a connection wall, the insertion of the needle still straight and with the angled ends correctly oriented into the end wall of the casing, the affixing of said needle to said end wall, the double bending of the needle until it adheres to the slanted connection wall and is aligned with the outer edge of the casing, and the final affixing of the needle at the end of the slanted connection wall.

Said connection wall or outer support wall can also be used as a grip for the new holder.

### BRIEF DESCRIPTION OF THE SEVERAL VIEWS OF THE DRAWINGS

The characteristics of the new process for the production of the new holder with needle bent at two points and of the product obtained therefrom will be better illustrated by the following description, with reference to the attached drawings as a non-restrictive example.

FIG. 1 shows a perspective view of a first embodiment the new holder, the holder comprising a casing (C) and a shaped needle (A).

FIG. 2 shows a side cross section of the holder of FIG. 1.

FIG. 3 shows a side cross-section of a second embodiment of the new holder.

### DETAILED DESCRIPTION OF THE INVENTION

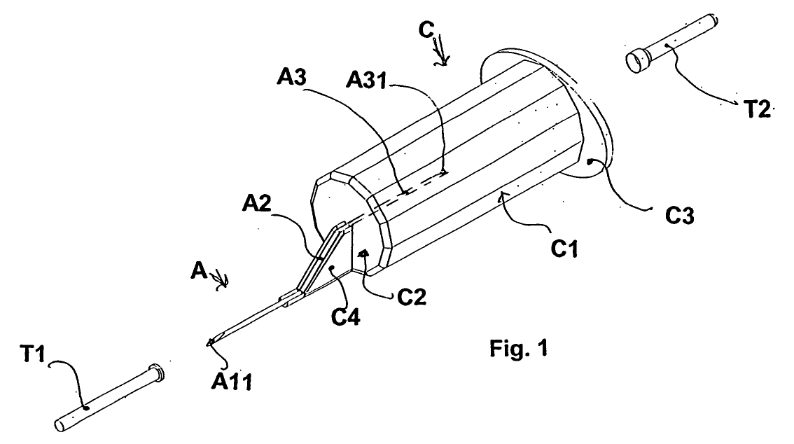

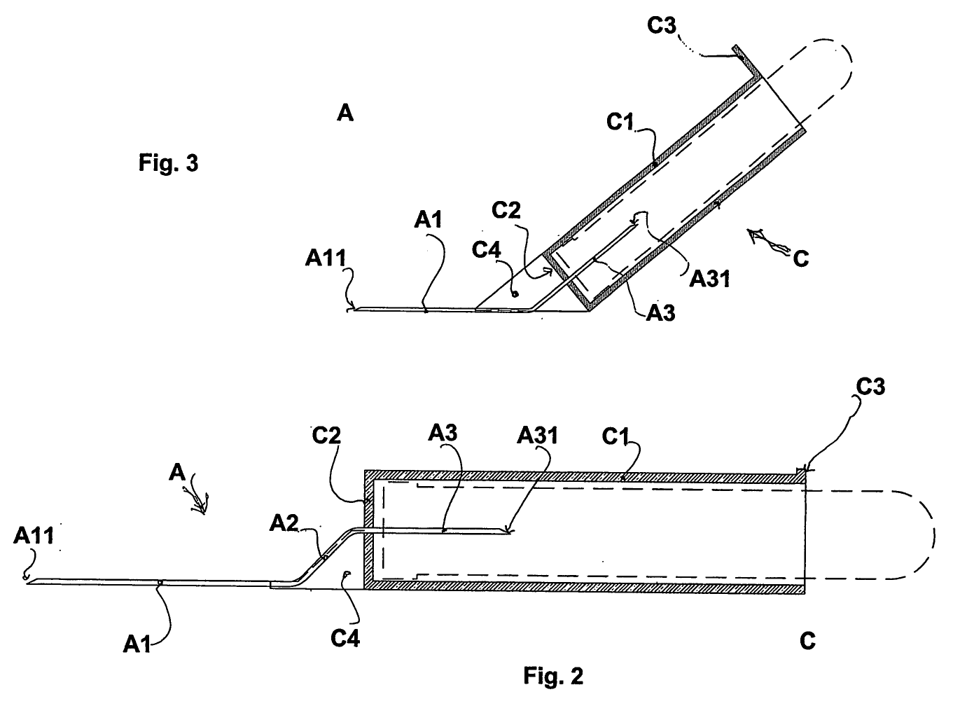

Turning first to FIGS. 1-2, there is shown a first embodiment of the invention. A shaped needle (A) consists of three consecutive sections (A1, A2, A3), of which the two extreme sections (A1, A3) are parallel and not lying on the same axis. The intermediate section (A2) connects said two extreme sections (A1, A3) and is slanted with respect to each of them.

Both of said extreme sections (A1, A3) have an angled or slanted tip (A11, A31), in order to facilitate the penetration into the skin and into a vein at one end and the perforation of, and penetration into, the seal cap of a vacuum test tube at the other end.

The casing (C) consists of a cylinder (C1) closed on the front side (C2) and provided with outer tabs (C3) at the other end.

On the front closing wall (C2) of the casing (C) the shaped needle (A) is applied, so that an end section (A3) of said needle (A) is positioned inside the casing (C), coaxial with the cylindrical part (C1) of the casing (C).

The other end (A1) of the shaped needle (A) is positioned outside the casing (C) and is substantially aligned with the lower outer cylindrical surface of the casing (C).

Due to the position of the shaped needle (A) with respect to the casing (C), its intermediate section (A2) is positioned outside the casing (C) and facing the front closing wall (C2) of the casing (C).

The tip of the needle (A) that is outside the casing (C) has the angled, or slanting side (A11) facing upwards or facing the center or the axis of the casing (C).

The casing (C) features a connection wall (C4) designed to house and connect the intermediate section (A2) of the needle (A) with the casing (C).

The lower edge of said connection wall (C4) is parallel to the lower outer section (A1) of the needle (A) and provides a sliding surface for the holder (C) during the introduction of the needle (A).

The contact surface between said connection wall (C4) and the needle (A), in particular the intermediate slanted section (A2) of the needle (A), is typically semicircular, preferably with a slight undercut, in order to correctly house and retain the needle (A).

This connection or support wall (C4) can also be used as a grip for the new holder.

Two caps (T1, T2) are provided, which can be applied to protect and cover the end sections (A1, A3) and the two tips (A1, A31) of the needle (A).

The casing (C), or part of it, can be imprinted with the color of the needle gauge international code.

FIG. 3 shows the side cross-section of a variant of the invention, in which the needle (A) is bent in two parts (A1a, A3a), more particularly, an end part (A3a) included inside the casing (C) and coaxial with it, and a second end part (A1a) positioned outside the casing (C) and slanted with respect to said casing (C).

The process for the production of said new holder with a needle bent at two points comprises only a few simple steps.

Initially the casing (C) is produced, preferably by molding a thermoplastic material, inclusive of the outer tabs (C3) and the slanted connection wall (C4). A hole for housing the needle (A) is provided on the front wall (C2).

The needle (A), linear and not yet bent, is inserted in said hole in said front wall (C2) of the casing, in such a way so to correctly position its end section (A3) inside the casing (C).

In this insertion phase, the needle (A) is already positioned with the slanted tip (A11) correctly positioned, more particularly, facing upwards with respect to the connection wall (C4).

At this point the needle (A) is affixed to the front wall (C2) of the casing (C), for example with an adhesive.

Subsequently the needle (A) is bent twice: initially the needle (A) is bent so that it is in contact with the connection wall (C4), more particularly, it is positioned in the semicircular housing of said wall (C4); a successive bending operation, aligns the end section (A1) of the needle (A) with the outer surface of the casing (C) at the end of connection wall (C4) and with the slanted surface of the connection wall (C4).

Lastly, the two sections (A1, A2) of the needle (A) outside of the casing (C) are affixed to the connection wall (C4), preferably by means of an adhesive, in correspondence with the end of the connection wall (C4) opposite casing (C).

The method described above for the production of the new holder with a needle bent at two points requires the simple molding of the casing (C) and the preparation of the needle (A) in straight form and with an angled tip (A11), the insertion of the needle (A) into the casing (C) and, lastly, the bending and joining of the needle (A) to the casing (C) and to the connection wall (C4).

The new holder constituted and produced as described above offers considerable advantages.

After removing the cap (T1) from the needle (A), the nurse grips the new holder by its cylindrical body (C1) or the wall or connection wall (C4), leaving the outer section (A1) of the needle (A) facing upwards.

Consequently, the new holder in the nurse's hand is already in the correct position for use, with the tip (A11) of the needle (A) already correctly facing upwards with respect to the patient's skin and vein.

The eccentric position of the needle (A1) with respect to the casing (C), and in particular its position aligned with the outer surface of the casing (C), allows the nurse to insert the needle (A) into the skin and vein with a minimum penetration angle.

The new holder, with its greatly reduced or nil penetration angle, significantly reduces the discomfort or pain caused to the patient and avoids possible vein perforation errors.

The new holder, with its greatly reduced penetration angle, facilitates the insertion of the needle (A) into the vein.

The new holder with the eccentric needle (A) allows the nurse to correctly insert the needle (A) into the vein with no need to take any particular precautions, such as raising the patient's skin or varying the penetration angle during penetration.

The reduced penetration angle of the new holder considerably reduces the risk of perforating the vein from side to side.

The new holder, having the needle section (A3) inside the casing (C) positioned coaxially with the casing, permits the use of the common and widespread vacuum test tubes with seal cap designed for a central perforation.

Therefore, with reference to the preceding description and the attached drawings, the following claims are put forth.

What is claimed is:

1. A process for the production of holders for interchangeable vacuum test tubes comprising:
(a) providing a tubular casing (C) designed to house a vacuum test tube for blood sampling, the casing comprising a side wall and a front wall (C2) with an opening for housing a needle, the casing further comprising a support wall (C4) substantially triangular in shape and extending from the front wall (C2), the support wall (C4) having with a first side aligned with the front wall (C2) and a second side aligned with the side wall, the first side being positioned between the opening and the side wall, the support wall further comprising a third side connecting the first and the second sides;
(b) partially inserting the needle (A) in straight shape through the opening in said casing (C), the needle being affixed to the front wall (C2), the needle (A) having an inner portion (A3) essentially coaxial with the casing (C) and housed inside the casing (C), the needle having an outer portion extending from the casing and ending with an angled tip facing in the direction of the longitudinal axis of the casing (C);
(c) causing a first bending of the outer portion of the needle (A) to slant it with respect to the axis of the casing (C) and to make it contact the third side of the support wall (C4);
(d) causing a second bending of the outer portion (A1) of the needle (A) to align the terminal outer portion of the needle (A) with the side wall of the casing (C); and
(e) affixing at least a part of the slanted outer portion of the needle to the third side of the support wall.

2. A holder for interchangeable vacuum test tubes comprising:
a casing (C) essentially tubular in shape, the casing (C) having a side wall and a closing wall (C2) at one end; and
a needle (A) bent in at least three portion, the two end portions (A1, A3) being parallel and not lying on the same axis, the intermediate portion (A2) connecting said end portions (A1, A3), the intermediate portion (A2) being external to said casing (C) and slanted in relation to the closing wall (C2),
wherein said needle (A) is applied to the closing wall (C2) so that one end portion (A3) of the needle (A) is inside the casing (C) and is coaxial with it,
wherein the other end portion (A1 1) of the needle is situated outside the casing (C) and substantially aligned with the side wall of the casing (C), and
wherein the closing wall (C2) comprises a support wall (C4) designed to house the intermediate portion (A2) of the needle (A) and to connect the needle with the casing (C), the support wall further providing a lower sliding surface for the casing (C) against a patient's limb during the introduction of the needle (A) into the limb.

3. A holder for interchangeable vacuum test tubes comprising:
a casing (C) essentially tubular in shape, the casing having a closing wall (C2) at one end;
a needle (A) bent in two portions (A1a, A3a),
wherein said needle (A) is applied to said closing wall (C2) of the casing (C), so that a portion (A3a) of the needle (A) is inside the casing (C) and coaxial with the casing,
wherein the other portion (A1a) of the needle (A) is outside the casing (C) and slanted with respect to the casing (C), and
wherein the closing wall (C2) is provided with a support wall (C4) designed to house the outer part (A1a) of the needle (A) and to connect it with the casing (C), the support wall further providing a lower sliding surface for the holder (C) during the introduction of the needle (A) into a patient's limb.

4. The holder for interchangeable vacuum test tubes of claim 2, wherein the support wall (C4) of the casing is provided with a semicircular seat suitable for securely housing the needle (A).

5. The holder for interchangeable vacuum test tubes of claim 2, wherein the end portion of the needle situated outside the casing has an end tip (A11) that is slanted and facing in the direction of the longitudinal axis of the casing (C).

6. The holder for interchangeable vacuum test tubes of claim 2, wherein the holder is further provided with at least one cap (T1) that can be applied to protect and cover the tip (A11) of the needle (A) situated outside the casing (C).

7. The holder for interchangeable vacuum test tubes of claim 2, wherein the exterior surface of the casing (C) bears one or more of a color or wording indicating the international code of the needle gauge.
